(19) United States
(12) Patent Application Publication
(10) Pub. No.: US 2006/0014266 A1
(43) Pub. Date: Jan. 19, 2006

(54) Purification of Arp2/3 complex and compositions containing purified Arp2/3 complex

(75) Inventors: Nenad Tomasevic, Foster City, CA (US); Roman Sakowicz, Foster City, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Cytokinetics, Inc., South San Francisco, CA (US)

(21) Appl. No.: 11/149,945

(22) Filed: Jun. 10, 2005

Related U.S. Application Data
(60) Provisional application No. 60/578,969, filed on Jun. 10, 2004.

(51) Int. Cl. C12N 9/64

(52) U.S. Cl. 435/226; 530/350; 530/416

(57) ABSTRACT

Methods for rapidly purifying Arp2/3 complex are provided. Compositions containing purified Arp2/3 complex that is characterized by having equal stoichiometry of the subunits are also disclosed.

### CROSS-REFERENCES TO RELATED APPLICATIONS

This application claims the benefit of U.S. Provisional Application No. 60/578,969, filed Jun. 10, 2004, which is incorporated herein by reference in its entirety for all purposes. This application is related to U.S. application Ser. No. ______, filed ______, which claims the benefit of U.S. Provisional Application Nos. 60/578,949, filed Jun. 10, 2004, and 60/673,444, filed Apr. 20, 2005, all of which are incorporated herein by reference in their entirety for all purposes. This application is also related to U.S. application Ser. No. ______, filed ______, which claims the benefit of U.S. Provisional Application No. 60/578,913, filed Jun. 10, 2004, both of which are incorporated herein by reference in their entirety for all purposes.

### BACKGROUND

Many cells utilize actin polymerization to perform a number of essential cellular processes. Eukaryotic cells, for example, use actin polymerization to regulate motility, change shape and to internalize extracellular materials via endocytosis and phagocytosis. Furthermore, a number of pathogens have evolved to subvert host cell actin assembly for the purposes of attachment, internalization and to spread from cell to cell.

These processes depend upon the rapid and localized assembly and disassembly of actin filaments. New filaments are created by nucleation of monomeric actin (Carson, M. et al. (1986) J. Cell Biol. 103:2707-2714; Chan, A. Y. et al. (1998) J. Cell Sci. 111:199-211), which refers to the initiation of actin polymerization from free actin monomers and is the rate-limiting step in the assembly of actin filaments. The very large kinetic barrier to nucleation indicates that regulation of the nucleation step may be critical to controlling actin polymerization in cells. Control of actin nucleation then is also likely important in regulating the foregoing key cellular functions in which actin polymerization plays an important role.

The actin nucleation machinery includes at least two key components: the Arp2/3 complex and one or more members from the family of nucleation promoting factors (NPFs). The Arp2/3 complex (or simply Arp2/3) is responsible for nucleating new actin filaments and cross-linking newly formed filaments into Y-branched arrays. In particular, the Arp2/3 complex is positioned at the Y-branch between the filaments and stabilizes the cross-link region. The Arp2/3 complex consists of six subunits in Saccharomyces cerevisiae and seven subunits in Acanthaemoeba castellanii and humans. These subunits are present in equal stoichiometry. The two largest subunits (50 and 43 kDa) are actin-related proteins in the Arp3 and Arp2 families, respectively. The name of the complex is thus named after these two subunits. The other five subunits in the human complex have molecular masses of approximately 40, 35, 21, 20 and 19 kDa, based upon sodium dodecyl sulfate-polyacrylamide gel electrophoresis studies and are referred to as p40, p35, p21, p20 and p19, respectively.

Arp2/3 by itself, however, possesses little activity. The complex must be bound by a NPF to become activated. Examples of such NPFs include Wiskott-Aldrich syndrome protein (WASP), a WASP homolog call N-WASP, and a family of proteins called suppressor of cAR (SCAR) (also referred to as the WASP family verprolin homologous (WAVE) proteins). See, for example, Welch, M. D. and Mullins, R. D. (2002) Annu. Rev. Cell Dev. Biol. 18:247-288; and Higgs, H. N. and Pollard, T. D. (2001) Annu. Rev. Biochem. 70:649-76, both of which are incorporated herein by reference in their entirety for all purposes.

NPFs themselves are also regulated. They are activated by the binding of upstream regulatory molecules. Examples of such regulatory proteins involved in the activation of WASP and N-WASP include 1) the Rho-family GTPase, Cdc42, 2) the acidic lipid, phosphatidylinositol-4,5-bisphosphate (PIP₂), 3) Src family tyrosine kinases, 4) Btk and Itk tyrosine kinases, and 5) syndapin 1. See, e.g., Higgs and Pollard, supra.

Methods for rapidly purifying Arp2/3 complex are needed to produce the protein for conducting additional experiments regarding the structure, function and regulation of Arp2/3 can be undertaken. The purification methods that have been developed to date, however, have various shortcomings. Certain methods, for instance, are quite time consuming, with some methods extending 3-5 days. This is problematic because the Arp2/3 complex is relatively unstable. Thus, some of the current purification schemes result in a complex in which the balanced stoichiometry between the subunits is lost and/or certain subunits are degraded. This has been found to be a particular problem with the p40 subunit. Many existing methods utilize chromatographic methods in which the complex is eluted with magnesium ion salts. Magnesium ion, however, is known to affect actin polymerization dramatically; thus, purification methods utilizing such elution procedures often require extensive dialysis to remove the magnesium ion before the protein complex can be used in studies involving actin polymerization. This is time-consuming and increases the probability that a degraded complex is obtained. Certain protocols also yield relatively small amounts of the Arp2/3 complex, have poor yields and/or result in relatively impure complex. Another problem with some procedures is that they involve a large number of column chromatography steps, which often require adjustment of sample parameters (e.g., pH, ion composition) before the sample can be loaded onto the column. In view of such shortcomings, there is thus a need for new methods of purifying this important complex.

### BRIEF DESCRIPTION OF THE DRAWINGS

FIG. 1 provides a schematic representation of an exemplary method for purifying the Arp2/3 complex from human platelets. As indicated, methods of this type can be completed within 18 hours, beginning with the initial processing of the platelets to obtain an extract that contains the complex until collection of the purified complex.

FIG. 2 presents an image of the results of a sodium dodecyl sulfate (SDS) polyacrylamide gel electrophoresis analysis illustrating the purity of the Arp2/3 complex following elution from the affinity column utilized in certain of the current purification procedures. The affinity column utilized in this purification included an affinity matrix to which was immobilized a GST-VCA-HIS fusion protein (i.e., a VCA region from WASP to which a glutathione-S-transferase and His6 tag are respectively fused to the amino and carboxyl terminus). The identity of the samples applied to each of the lanes is as follows: Lane 1: Protein standards; Lane 2: Arp2/3 complex purified from bovine thymus, showing separation of the complex into the seven different subunits (Arp3, Arp2, p40, p35, p21, p20 and p19); and Lanes 3-9: Fractions eluted from the affinity column. The asterisk (*) alongside the second uppermost band in lane 7 indicates the position of citrate synthase.

FIG. 3 is a plot showing the extent of actin polymerization in the presence (line 1) and absence (line 2) of GST-VCA and Apr2/3 complex as a function of time. The vertical axis is in units of Relative Fluorescence Units (RFU). Details of the assay are provided in Example 4.

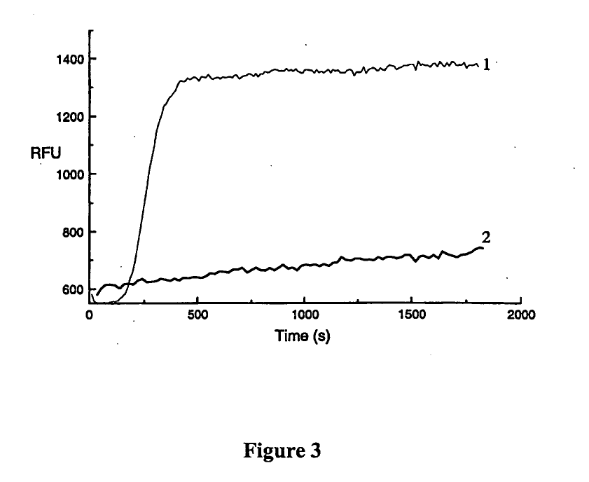

FIG. 4 is a schematic representation of the major domains of NPFs from the WASP and N-WASP family.

### DETAILED DESCRIPTION

1. Definitions

All technical and scientific terms used herein have the meaning commonly understood by a person skilled in the art to which this invention belongs, including the definitions provided herein. The following references provide one of skill with a general definition of many of the terms used in this invention: Singleton et al., DICTIONARY OF MICROBIOLOGY AND MOLECULAR BIOLOGY (2d ed. 1994); THE CAMBRIDGE DICTIONARY OF SCIENCE AND TECHNOLOGY (Walker ed., 1988); THE GLOSSARY OF GENETICS, 5TH ED., R. Rieger et al. (eds.), Springer Verlag (1991); and Hale & Marham, THE HARPER COLLINS DICTIONARY OF BIOLOGY (1991).

An “Arp2/3 complex” refers to a protein complex that in conjunction with a nucleation promoting factor (e.g., WASP, N-WASP, SCAR/WAVE) or fragment or variant thereof can nucleate and cross-link monomeric actin filaments into a cross-linked network (see, e.g., Higgs, H. N. (2001) Annu. Rev. Biochem. 70:649-76). The term encompasses complexes having such activity from both mammalian and non-mammalian sources. The complex from at least mammalian systems typically includes 7 subunits: Arp3, Arp2, p40, p35, p21, p20 and p19. In an alternative nomenclature sometimes used in the literature, the subunits are referred to as ACTR2 (Arp2), ACTR3 (Arp3), ARPC1, ARPC2, ARPC3, ARPC4 and ARPC5. Arp2/3 complexes from certain non-mammalian sources (e.g., Saccharomyces cerevisiae) have six rather than seven subunits. The term also includes complexes that include fragments and variants of one or more of the component subunits so long as the complex retains its nucleation and cross-linking activity.

The term “exchanger” refers to an ion exchange chromatography material, which typically includes an insoluble matrix with charged groups linked (e.g., covalently) to the matrix. The term includes ion exchange material that is included in a column for use in column chromatography or simply in a container for use in batch methods.

The term “equivalent,” “equivalent exchanger” and other like phrases when used with respect to a reference ion exchange material generally refers to other ion exchangers that satisfy two primary criteria: 1) the useable pH range of the ion exchange materials overlap, such that the exchange materials have similar binding characteristics (e.g., binding capacity); and 2) the ion exchange materials can achieve comparable resolution (e.g., both resolve different proteins that elute closely in time with comparable efficiency). The term encompasses exchangers in which (i) the charged group of the exchangers are the same but the matrix to which the groups are bound differ, (ii) the exchangers share the same matrix but contain different ionizable groups, and (iii) the exchangers are made of different matrix materials and different ionizable groups, provided the two criteria specified above are satisfied. Equivalents to a DEAE Sepharose exchanger include other weak anion exchangers. Equivalents to Q-Sepharose include various strong anion exchangers that are known in the art.

The terms “nucleic acid,” “polynucleotide,” and “oligonucleotide” are used interchangeably and refer to a deoxyribonucleotide or ribonucleotide polymer in either single-, double, or triple-stranded form. For the purposes of the present disclosure, these terms are not to be construed as limiting with respect to the length of a polymer. The terms can encompass known analogues of natural nucleotides, as well as nucleotides that are modified in the base, sugar and/or phosphate moieties. In general, an analogue of a particular nucleotide has the same base-pairing specificity; i.e., an analogue of A will base-pair with T. The terms additionally encompass nucleic acids containing known nucleotide analogs or modified backbone residues or linkages, that are synthetic, naturally occurring, and non-naturally occurring and that have similar binding properties as the reference nucleic acid. Examples of such analogs include, without limitation, phosphorothioates, phosphoramidates, methyl phosphonates, chiral-methyl phosphonates, 2-O-methyl ribonucleotides, and peptide-nucleic acids (PNAs).

“Polypeptide” and “protein” are used interchangeably herein and include a molecular chain of amino acids linked through peptide bonds. The terms do not refer to a specific length of the product. Thus, “peptides,” “oligopeptides,” and “proteins” are included within the definition of polypeptide. The terms include post-translational modifications of the polypeptide, for example, glycosylations, acetylations, phosphorylations and the like. In addition, protein fragments, analogs, mutated or variant proteins, fusion proteins and the like are included within the meaning of polypeptide.

A “fusion protein” or “fusion polypeptide” is a molecule in which two or more protein subunits are linked, typically covalently. The subunits can be directly linked or linked via a linking segment. An exemplary fusion protein is one in which a domain from a nucleation promoting factor (e.g., the CA or VCA region) is linked to one or more purification tags (e.g., glutathione-S-transferase, His6, an epitope tag, and calmodulin binding protein).

The term “operably linked” or “operatively linked” is used with reference to a juxtaposition of two or more components (e.g., protein domains), in which the components are arranged such that each of the components function normally and allow the possibility that at least one of the components can mediate a function that is exerted upon at least one of the other components. By way of illustration, a transcriptional regulatory sequence (e.g., a promoter) is operably linked to a coding sequence if the transcriptional regulatory sequence controls the level of transcription of the coding sequence in response to the presence or absence of one or more transcriptional regulatory factors. With respect to fusion proteins or polypeptides, the terms can refer to the fact that each of the components performs the same function in the linkage to the other component as it would if it were not so linked. For example, in a fusion protein in which the VCA region of a nucleation promoting factor is fused to a glutathione-S-transferase (GST) tag, these two elements are considered to be operably linked if the VCA region can still bind to and activate Arp2/3 and the GST tag can bind to glutathione (e.g., the glutathione on a glutathione Sepharose matrix).

The terms “identical” or percent “identity,” in the context of two or more nucleic acids or polypeptides, refer to two or more sequences or subsequences that are the same or have a specified percentage of nucleotides or amino acid residues that are the same, when compared and aligned for maximum correspondence, as measured using a sequence comparison algorithm such as those described below for example, or by visual inspection.

The phrase “substantially identical” or “substantial sequence identity,” in the context of two nucleic acids or polypeptides, refers to two or more sequences or subsequences that have at least 75%, preferably at least 85%, more preferably at least 90%, 95% or higher nucleotide or amino acid residue identity, when compared and aligned for maximum correspondence, as measured using a sequence comparison algorithm such as those described below for example, or by visual inspection. Preferably, the substantial identity exists over a region of the sequences that is at least about 10, 20, 30, 40, 50 or 60 residues in length, in some instances over a longer region such as 60-80 amino acids, and in other instances over a region of at least about 90-100 nucleotides or amino acid residues. And, in still other instances, the sequences are substantially identical over the full length of the sequences being compared, such as the coding region of a nucleotide for example.

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Natl. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wisc.), or by visual inspection [see generally, Current Protocols in Molecular Biology, (Ausubel, F. M. et al., eds.), John Wiley & Sons, Inc., New York (1987-1999, including supplements such as supplement 46 (April 1999)]. Use of these programs to conduct sequence comparisons are typically conducted using the default parameters specific for each program.

Another example of algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra.). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always>0) and N (penalty score for mismatching residues; always<0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. For identifying whether a nucleic acid or polypeptide is within the scope of the invention, the default parameters of the BLAST programs are suitable. The BLASTN program (for nucleotide sequences) uses as defaults a word length (W) of 11, an expectation (E) of 10, M=5, N=−4, and a comparison of both strands. For amino acid sequences, the BLASTP program uses as defaults a word length (W) of 3, an expectation (E) of 10, and the BLOSUM62 scoring matrix. The TBLATN program (using protein sequence for nucleotide sequence) uses as defaults a word length (W) of 3, an expectation (E) of 10, and a BLOSUM 62 scoring matrix. (See Henikoff & Henikoff, Proc. Natl. Acad. Sci. USA 89:10915 (1989)).

In addition to calculating percent sequence identity, the BLAST algorithm also performs a statistical analysis of the similarity between two sequences (see, e.g., Karlin & Altschul, Proc. Natl. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001.

Another indication that two nucleic acid sequences are substantially identical is that the two molecules hybridize to each other under stringent conditions. “Bind(s) substantially” refers to complementary hybridization between a probe nucleic acid and a target nucleic acid and embraces minor mismatches that can be accommodated by reducing the stringency of the hybridization media to achieve the desired detection of the target polynucleotide sequence. The phrase “hybridizing specifically to” refers to the binding, duplexing, or hybridizing of a molecule only to a particular nucleotide sequence under stringent conditions when that sequence is present in a complex mixture (e.g., total cellular) DNA or RNA.

A further indication that two nucleic acid sequences or polypeptides are substantially identical is that the polypeptide encoded by the first nucleic acid is immunologically cross reactive with the polypeptide encoded by the second nucleic acid, as described below.

“Conservatively modified variations” of a particular polynucleotide sequence refers to those polynucleotides that encode identical or essentially identical amino acid sequences, or where the polynucleotide does not encode an amino acid sequence, to essentially identical sequences. Because of the degeneracy of the genetic code, a large number of functionally identical nucleic acids encode any given polypeptide. For instance, the codons CGU, CGC, CGA, CGG, AGA, and AGG all encode the amino acid arginine. Thus, at every position where an arginine is specified by a codon, the codon can be altered to any of the corresponding codons described without altering the encoded polypeptide. Such nucleic acid variations are “silent variations,” which are one species of “conservatively modified variations.” Every polynucleotide sequence described herein which encodes a polypeptide also describes every possible silent variation, except where otherwise noted. One of skill will recognize that each codon in a nucleic acid (except AUG, which is ordinarily the only codon for methionine) can be modified to yield a functionally identical molecule by standard techniques. Accordingly, each “silent variation” of a nucleic acid which encodes a polypeptide is implicit in each described sequence.

A polypeptide is typically substantially identical to a second polypeptide, for example, where the two peptides differ only by conservative substitutions. A “conservative substitution,” when describing a protein, refers to a change in the amino acid composition of the protein that does not substantially alter the protein's activity. Thus, “conservatively modified variations” of a particular amino acid sequence refers to amino acid substitutions of those amino acids that are not critical for protein activity or substitution of amino acids with other amino acids having similar properties (e.g., acidic, basic, positively or negatively charged, polar or non-polar, etc.) such that the substitutions of even critical amino acids do not substantially alter activity. Conservative substitution tables providing functionally similar amino acids are well-known in the art. See, e.g., Creighton (1984) Proteins, W.H. Freeman and Company. In addition, individual substitutions, deletions or additions which alter, add or delete a single amino acid or a small percentage of amino acids in an encoded sequence are also “conservatively modified variations.”

The term “stringent conditions” refers to conditions under which a probe or primer will hybridize to its target subsequence, but to no other sequences. Stringent conditions are sequence-dependent and will be different in different circumstances. Longer sequences hybridize specifically at higher temperatures. Generally, stringent conditions are selected to be about 5° C. lower than the thermal melting point (Tₘ) for the specific sequence at a defined ionic strength and pH. In other instances, stringent conditions are chosen to be about 20° C. or 25° C. below the melting temperature of the sequence and a probe with exact or nearly exact complementarity to the target. As used herein, the melting temperature is the temperature at which a population of double-stranded nucleic acid molecules becomes half-dissociated into single strands. Methods for calculating the Tₘ of nucleic acids are well known in the art (see, e.g., Berger and Kimmel (1987) Methods in Enzymology, vol. 152: Guide to Molecular Cloning Techniques, San Diego: Academic Press, Inc. and Sambrook et al. (1989) Molecular Cloning: A Laboratory Manual, 2nd ed., vols. 1-3, Cold Spring Harbor Laboratory), both incorporated herein by reference. As indicated by standard references, a simple estimate of the Tm value can be calculated by the equation: Tₘ=81.5+0.41 (% G+C), when a nucleic acid is in aqueous solution at 1 M NaCl (see e.g., Anderson and Young, “Quantitative Filter Hybridization,” in Nucleic Acid Hybridization (1985)). Other references include more sophisticated computations which take structural as well as sequence characteristics into account for the calculation of Tₘ. The melting temperature of a hybrid (and thus the conditions for stringent hybridization) is affected by various factors such as the length and nature (DNA, RNA, base composition) of the probe and nature of the target (DNA, RNA, base composition, present in solution or immobilized, and the like), and the concentration of salts and other components (e.g., the presence or absence of formamide, dextran sulfate, polyethylene glycol). The effects of these factors are well known and are discussed in standard references in the art, see e.g., Sambrook et al., Molecular Cloning: A Laboratory Manual, 3rd edition, Cold Spring Harbor Press, N.Y., (2001); Current Protocols in Molecular Biology, (Ausubel, F. M. et al., eds.), John Wiley & Sons, Inc., New York (1987-1993). Typically, stringent conditions will be those in which the salt concentration is less than about 1.0 M Na ion, typically about 0.01 to 1.0 M Na ion concentration (or other salts) at pH 7.0 to 8.3 and the temperature is at least about 30° C. for short probes or primers (e.g., 10 to 50 nucleotides) and at least about 60° C. for long probes or primers (e.g., greater than 50 nucleotides). Stringent conditions can also be achieved with the addition of destabilizing agents such as formamide.

The term “isolated,” “purified” or “substantially pure” means an object species (e.g., an Arp2/3 complex) is the predominant macromolecular species present (i.e., on a molar basis it is more abundant than any other individual species in the composition), and preferably the object species comprises at least about 50 percent (on a molar basis) of all macromolecular species present. Generally, an isolated, purified or substantially pure Arp2/3 complex or nucleic acid will comprise more than 80 to 90 percent of all macromolecular species present in a composition. Most preferably, the object species is purified to essential homogeneity (i.e., contaminant species cannot be detected in the composition by conventional detection methods) wherein the composition consists essentially of a single macromolecular species.

Various biochemical and molecular biology methods are well known in the art. For example, methods of isolation and purification of nucleic acids are described in detail in WO 97/10365, WO 97/27317, Chapter 3 of Laboratory Techniques in Biochemistry and Molecular Biology: Hybridization With Nucleic Acid Probes, Part 1. Theory and Nucleic Acid Preparation, (P. Tijssen, ed.) Elsevier, N.Y. (1993); Chapter 3 of Laboratory Techniques in Biochemistry and Molecular Biology: Hybridization With Nucleic Acid Probes, Part 1. Theory and Nucleic Acid Preparation, (P. Tijssen, ed.) Elsevier, N.Y. (1993); and Sambrook et al., Molecular Cloning: A Laboratory Manual, 3rd edition, Cold Spring Harbor Press, N.Y., (2001); Current Protocols in Molecular Biology, (Ausubel, F. M. et al., eds.) John Wiley & Sons, Inc., New York (1987-1993). Large numbers of tissue samples can be readily processed using techniques known in the art, including, for example, the single-step RNA isolation process of Chomczynski, P., described in U.S. Pat. No. 4,843,155.

II. Overview

Methods for rapidly purifying the Arp2/3 complex in high purity and yield from a variety of source materials are provided. The methods have been designed to facilitate the rapid recovery of stable and high purity complex. The rapid recovery of the complex is important to avoid disassociation of component subunits from the complex and/or degradation or modification of the subunits. In some existing purification methods, the equal stoichiometry of the constituent subunits is lost and/or some subunits degraded. Degradation of the p40 subunit in particular, and the other subunits to a lesser extent, has been found to be problematic with some purification methods.

Decreases in processing time are made possible, in part, by the judicious use of a series of column chromatography methods in which one anion exchange column binds some contaminating proteins, whereas the Arp2/3 complex flows directly through without binding. The eluant from this first column can then optionally be applied directly to a second anion exchange column without any prior sample processing. The chromatography procedures in the purification can also be conducted with solutions that contain low magnesium ion concentrations or no magnesium ion. This is important because magnesium ion significantly affects actin polymerization. Thus, any studies to be conducted with the Arp2/3 complexes containing high magnesium ion concentrations must first involve removing magnesium ion (e.g., by time-consuming dialysis), which increases the risk of protein degradation. Inclusion of an affinity chromatography step also facilitates rapid processing and selective purification. The purification methods that are disclosed can also be automated, thereby decreasing purification time and simplifying the process.

Whereas certain other purification methods take 3-5 days to complete, certain purification methods that are described herein can typically be completed in less than 24 hours and, in some instances, in 18 hours or less. The methods that are provided can also be utilized with essentially any source of Arp2/3, thus making the methods quite versatile. The limited number of processing steps involved also means that high recovery levels can be achieved.

The resulting protein complex is of high purity, and the equal stoichiometry and the structural integrity of the subunits is maintained. Thus, high purity Arp2/3 compositions in which the stoichiometry of the constituent subunits of the complex is balanced and in which the subunits are not degraded are also provided. Finally, affinity chromatography material bearing immobilized affinity ligands for purifying Arp2/3 complex are also described herein.

III. Purification Procedures

A. Generally

The purification procedures that are provided typically involve four primary steps: First, a sample is provided that includes Arp2/3 complex. This can be done by lysing cells such as human platelets that contain relatively high amounts of the Arp2/3 complex. Second, the sample containing Arp2/3 complex is loaded onto a first anion exchange column (e.g., DEAE or equivalent) under conditions in which some contaminating proteins bind to the exchanger but the complex does not. Third, the eluate from the first anion exchange column is applied to a second anion exchange column (e.g., Q-Sepharose or equivalent), wherein the Arp2/3 complex is initially bound and then eluted from the column using a salt gradient. Finally, the active fractions collected from the second ion exchange column are applied to an affinity column matrix under conditions in which Arp2/3 is bound. It is subsequently eluted after first eluting contaminating proteins. The first and second ion exchange columns can be run in an automated and continuous process in which eluate from the first column is applied directly to the second.

The entire purification process beginning with an Arp2/3 source (e.g., outdated platelets) to the final purified protein complex that is ready for storage can often be conducted within 24 hours, and typically within 18-30 hours, such as within 18-24 hours. Typical protein recovery is about 50-60% of the Arp2/3 present in the initial cellular extract. The resulting Arp2/3 complex can be at least 80, 90 or 95% pure; homogeneous protein solutions can also be obtained.

The various processing steps, including the chromatography processes, can be performed in the presence of minimal magnesium ion. The magnesium concentration is typically not more than 1-10 mM (e.g., 1 or 2 mM). Some purification methods are conducted in the absence of detectable magnesium ion.

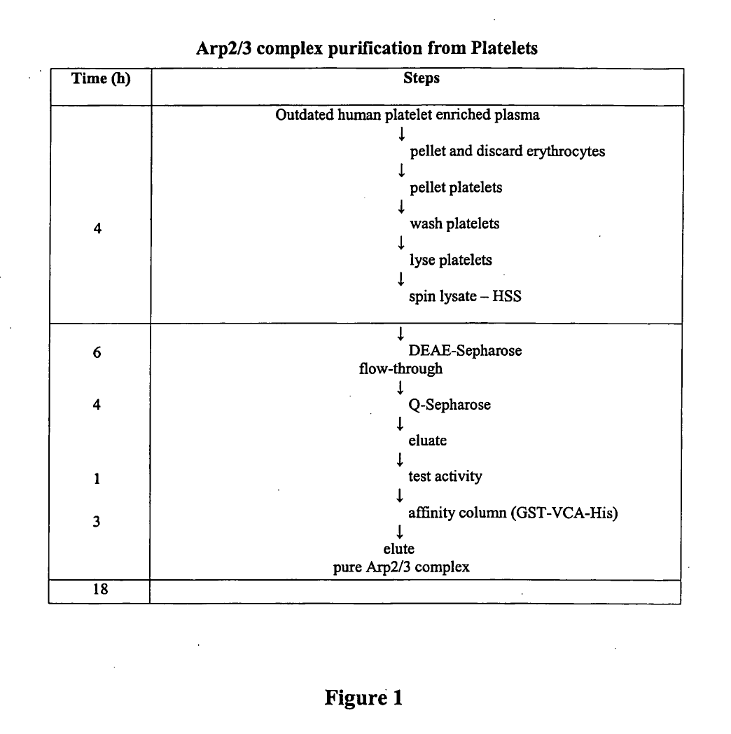

The overall general process is represented in somewhat greater detail in FIG. 1 and described more fully in the following sections.

B. Sources of Arp2/3

The purification methods that are provided herein can be utilized with essentially any source that contains Arp2/3 complex. Human platelets are a useful source of Arp2/3 for a number of reasons, including: 1) the relatively high abundance of the Arp2/3 complex in platelets; 2) the relatively low level of proteases present in a crude platelet extract; 3) the human origin of the protein complex, 4) the relatively low total protein complexity in platelets, which minimizes the amount of contaminating protein present; 5) the soft cellular membrane of platelets that facilitates preparation of a cellular extract; and 6) the fact that outdated platelets can be obtained inexpensively. For a further discussion of human platelets as a source of the Arp2/3 complex, see, e.g., Welch, M. D. et al. (1997) Nature 385:265. Platelets from other sources can also be utilized, such as chicken, bovine and porcine platelets.

Despite the foregoing advantages associated with human platelets, compositions containing the Arp2/3 complex can be obtained from a number of other sources as well. Other suitable sources include Acanthanmoeba castellanii (see, e.g., Kelleher, J. F., et al. (1998) Meth. Enzymol. 298:42-51), Saccharomyces cerevisiae (Winter, D., et al. (1997) Curr. Biol. 7:519) and bovine brain (see, e.g., Egile, C., et al. (1999) J. Cell Biol. 146:1319-1332) (each of these three references being incorporated herein by reference in its entirety for all purposes). Arp2/3 can also be obtained from bovine and pig thymus or spleen.

C. Production of Arp2/3 Extract

As described in greater detail in Example 1 and summarized in FIG. 1, a crude extract containing the Arp2/3 complex can be obtained relatively quickly from cells containing Arp2/3. When an extract is to be prepared from platelets, the method generally involves: 1) spinning down platelets by centrifugation at a relatively low speed (e.g., 400 g) to precipitate out the red blood cells, thus leaving the platelets suspended in the supernatant; 2) centrifuging the suspended platelets at higher speed (e.g., 1200 g) to form a pellet of platelets; 3) resuspending the platelets in a relatively small volume (e.g., 5-10 ml) of a buffered solution (pH 7.3-7.8, e.g., 7.5) that contains protease inhibitors to prevent proteolytic degradation of the Arp2/3 complex; 4) lysing the resuspended platelets to form a lysate that contains the Arp2/3 complex; 5) spinning the lysate via centrifugation to precipitate out cellular debris formed during the lysis step; and 6) collecting the supernatant obtained from step 5 to obtain an extract that contains the Arp2/3 complex.

The extract that is obtained can be loaded directly onto the first anion exchange column. Alternatively, the extract can be stored for future use. One storage option is to drop freeze the extract in liquid nitrogen. For each 10 L of outdated human platelets, it is typical to obtain about 700 ml of a lysate that has a total protein concentration of about 20 mg/ml.

Other extraction processes can also be utilized to prepare the extract that is utilized in certain purification methods. Additional methods for preparing an extract from human cells are discussed by Welch and Mitchison (Meth. Enzymology 298:52-61, 1988) and Higgs, H. N., et al. (Biochemistry 38:15212-15222, 1999), both of which are incorporated herein by reference in their entirety for all purposes. Methods for preparing extracts from Acanthamoeba castellanii are discussed, for example, by Kelleher, J. F., et al. (Meth. Enzymology 298:42-51, 1988), and Dayel, M. J., et al. (Proc. Natl. Acad. Sci. 98:14871-14876, 2001), both of which are incorporated herein by reference in their entirety for all purposes.

D. First Anion Exchange Column

The extract containing the Arp2/3 complex is typically loaded onto a first column of anion exchange material. The anion exchange material and the elution conditions are chosen such that most of the Arp2/3 complex present in the extract passes through the exchange material without binding. Certain other contaminating proteins, however, do bind the exchanger under these conditions, thus resulting in some purification of the Arp2/3 complex.

The anion exchanger utilized in this step of the purification is generally one in which the charged group of the exchanger matrix or resin is a tertiary amine or related group that is positively charged at neutral pH. One suitable charged group satisfying these criteria is the diethylaminoethyl (DEAE) group. Thus, a common anion exchange material for this purification step is any of the commercially available DEAE chromatography materials or equivalents thereof (e.g., other weak anion exchangers). Specific examples of such materials include, but are not limited to, DEAE-cellulose materials such as DEAE Sepharose and DEAE-Sephacel. These materials are readily available from a number of commercial manufacturers, including Amersham Biosciences.

Since this anion exchanger column is run under conditions in which the Arp2/3 complex flows through the column, the flowthrough is collected. The column is then typically washed (e.g., about 2 column volumes) to maximize recovery of the Arp2/3 complex. Typically, the Arp2/3 complex is applied and eluted at a pH that is between 7.8-8.2. In certain methods, the pH of the elution buffer is greater than 8. The elution buffer typically contains a buffer, metal ions (e.g., Mg²⁺ and/or K⁺), ATP, protein stabilizers (e.g., glycerol and a reducing agent such as DTT), and a protease inhibitor. The composition of an exemplary elution buffer (Buffer A) is 10 mM Tris, pH 8.0 (room temperature), 1 mM DTT, 1 mM MgCl₂, 30 mM KCl, 0.2 mM ATP, 1 mM EGTA/KOH, 2% glycerol (v/v). To this buffer 1 mM PMSF, and 2 tablets of protease inhibitors (Roche)/L is typically included. Further details regarding this first anion exchange purification process are provided in Example 3

E. Second Anion Exchange Column

The flowthrough and wash solution collected from the first column is applied to a second anion exchange column that is run under conditions such that the Arp2/3 complex binds to the exchanger. The first and second anion exchange columns can be run so the solution collected from the first column is directly applied to the second anion exchange column without any sample preparation (e.g., sample concentration, or adjustment of salt concentration or pH) prior to loading.

Various types of anion exchange material can be utilized in the second column. One typical material is one that includes a quaternary amine or an equivalent group. Specific examples of suitable anion exchange material for the second column include Q Sepharose and equivalents thereof, such as other strong anion exchangers.

Once the solution from the first column has been applied, the second anion exchange column is typically washed with about 5-10 column volumes of elution buffer to elute non-bound proteins. The elution buffer can vary, but in some instances is the same as the elution buffer as that used in the first column, optionally without the protease inhibitors or PMSF. Thus, an exemplary buffer is the Buffer A described with respect to the first anion exchange chromatography procedure.

Bound Arp2/3 complex is subsequently eluted with a salt gradient (e.g., KCl or NaCl) having a beginning concentration of about 20, 30 or 40 mM (e.g., 30 mM) and a final salt concentration of about 270, 290, 300, 310 or 320 mM (e.g., 300 mM). One exemplary gradient that gives good results in conjunction with a Q Sepharose column is a 30-300 mM gradient of KCl in Buffer A. Arp2/3 under these conditions typically elutes at about 250 mM KCl. These conditions have been found to be sufficiently gentle so as not to disrupt the equal stoichiometry of the subunits of the complex. The gradient is typically run using the same buffer solution as during the wash (e.g., Buffer A). Gradients that have similar ionic strengths to those just described can also be utilized in some instances.

Fractions of the eluate are collected (typically 1-3 ml fractions, such as 2 ml fractions) and then assayed to identify those fractions containing Arp2/3. Various assays can be utilized to detect Arp2/3 (see below). Active fractions are pooled for further purification by affinity chromatography.

Although typically the first anion exchange column (e.g., DEAE column) is run before the second column (e.g., Q Sepharose) in some instances the order can be reversed.

F. Affinity Chromatography

The fractions containing Arp2/3 collected from the second ion exchange chromatography step are further purified by affinity chromatography. The affinity matrix for the affinity chromatography step generally includes a support and an affinity ligand directly or indirectly linked to the support. The affinity ligand includes an Arp2/3 binding domain, which generally includes a region from the C-terminus of a nucleation promoting factor (NPF) that is sufficient for Arp2/3 binding. The affinity ligand can also optionally include one or more tags at the amino and/or carboxyl end of the Arp2/3 binding domain. Further details on options for the composition of the affinity ligand are provided below.

Once the collected fractions have been applied to the affinity column, the column is generally washed with about 3-7 (e.g., 5) column volumes of buffer to remove unbound proteins. A typical wash solution is Buffer A with 30 mM KCl; equivalent wash solutions can be used instead. Purified Arp2/3 complex is subsequently eluted by raising the salt concentration until the Arp2/3 complex is displaced from the affinity matrix. Using affinity matrices of the type described below, a salt concentration of about 230-270 mM salt (e.g., 250 mM KCl in Buffer A) is sufficient to elute Arp2/3 complex. Under these conditions the equal stoichiometry of the Arp2/3 subunits can be maintained.

Fractions containing Arp2/3 are identified using the assays such as those described below and collected. The active fractions can be concentrated using conventional means. The salt concentration of the concentrated solution is also typically adjusted to about 30 mM KCl or equivalent. The purified protein can be stored in 30% glycerol (v/v) at −20° C. Further details regarding the preparation of affinity matrices and columns are provided in the examples below.

I. Affinity Ligand

The Arp2/3 binding domain of an affinity ligand generally refers to a protein having an amino acid sequence from the carboxyl terminal region of a NPF, or a protein having substantial sequence identity with such a protein. The Arp2/3 binding domain exists in a constitutively active form. As described in greater detail below, “constitutively active” as used in this context means 1) that the binding domain exists in active form in which there is no intramolecular binding that inhibits the ability of the binding domain to bind Arp2/3, and 2) that the binding domain can bind Arp2/3 in the absence of upstream regulatory molecules (e.g., Cdc42 and PIP₂).

There are a number of NPFs from which the Arp2/3 binding domain can be obtained. Examples of suitable NPFs include, but are not limited to, (1) WASP, (2) N-WASP, (3) the SCAR/WAVE family of proteins and (4) Act A protein from Listeria monocytogenes (see Table 1; see also Welch and Mullins (2002) Annu. Rev. Cell Dev. Biol. 18:247-88; and Higgs and Pollard (2001) Ann. Rev. Biochem. 70:649-76, which are incorporated herein by reference in their entirety for all purposes). GenBank accession numbers for the protein sequences of these proteins are listed in Table 1. This table also lists SEQ ID NOs: that provide exemplary amino acid sequences for these NPFs.

A common feature of all these NPFs is the CA region. As used herein, the “CA region” has its generally meaning in the art and refers to a C-terminal region that is conserved among NPFs and that can bind Arp2/3. The CA region includes a short cofilin homology segment (C) of basic amino acids and a short segment of acidic amino acids (A) and in general includes about 30-50 amino acids from the C-terminus of a NPF. Table 1 indicates the general region corresponding to the CA sequence for those NPFs listed in the table. It should be understood, however, that the CA region as defined herein can also include or omit 1-10 amino acids from either the amino or carboxyl end of the region as defined in Table 1. The CA region as defined herein, however, does not extend into the V domain of those proteins that have a VCA region (see below). Given its ability to bind Arp2/3,the Arp2/3 binding domain of some affinity ligands includes a CA region from a NPF.

The WASP, N-WASP and the SCAR/WAVE family of proteins are a subgroup of NPFs in which the CA region is part of a larger VCA region (also sometimes referred to in the art as the WWA or simply WA region). A “VCA region” as used herein has its general meaning in the art and typically includes about 70-100 amino acids from the C-terminal region of WASP, N-WASP, SCAR/WAVE and related proteins. The VCA region consists of one or two WASP homology 2 motifs that make up the V or W region. This V or W region in turn is joined to the CA region, with the C region serving as a linker between the V and A regions. General regions corresponding to the VCA region of WASP, N-WASP and SCAR/WAVE are listed in Table 1. It should be recognized, however, that these regions are exemplary and that the VCA region as defined herein can omit or include 1-10 amino acids from the amino and/or carboxyl ends of these regions as defined in Table 1. Because it can bind Arp2/3, the Arp2/3 binding domain of some affinity ligands includes the VCA region from a NPF.

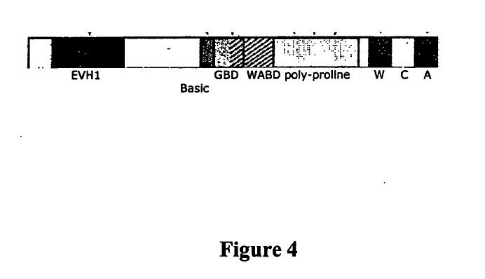

The Arp2/3 binding domain of the affinity ligand can include just the CA or VCA region of a NPF, or a larger region of the NPF that includes the CA or VCA region, provided, as noted above, the binding domain is constitutively active. The issue of constitutive activity arises because intermolecular interactions between certain domains in WASP and N-WASP keep the proteins in an inactive form until an upstream regulatory molecule binds to WASP or N-WASP and activates it. WASP and N-WASP, for example, share a number of other domains that are the binding sites for upstream regulators of WASP and N-WASP (e.g., Cdc42, PIP₂ and Nck). As illustrated in FIG. 4, these regulatory domains, listed in the amino to carboxyl direction, include a WH-1 domain (sometimes called a EVH1 domain), a basic (B) domain, a GTPase binding domain (GBD), and a poly-proline region (PolyPro) that is linked to the VCA region.

Both WASP and N-WASP are thought normally to exist in an inactive state due to intramolecular binding between the GBD and the cofilin binding domain (C), which blocks the site involved in Arp2/3 binding. The binding of upstream activators to WASP and N-WASP disrupts this intramolecular interaction, thereby freeing the CA region for binding with Arp2/3. See, e.g., Welch and Mullins (2002) Annu. Rev. Cell Dev. Biol. 18:247-88; and Higgs and Pollard (2001) Ann. Rev. Biochem. 70:649-76.

Given this interaction, if the Arp2/3 binding domain includes additional amino acid sequence from a NPF that is N-terminal to the VCA region, the Arp2/3 binding domain is generally a constitutively active form in which the intramolecular binding between the GBD and the C region cannot occur. Such constitutively active domains, for instance, typically are substantially free of GBD, for example. “Substantially free” when used with respect to the GBD means that sufficient GBD has been deleted such that the Arp2/3 binding domain is constitutively active and that a GTPase need not be present to disrupt any intramolecular binding between the GBD and the Arp2/3 binding site. It should be appreciated that Arp2/3 binding domains that include just the CA domain or VCA domain or fragments thereof are constitutively active by definition because they do not include the GBD.

Examples of suitable Arp2/3 binding domains thus include, but are not limited to: 1) the CA region from a NPF (see, e.g., Table 1), 2) the VCA region from a NPF (see, e.g., Table 1) or a fragment thereof that can bind Arp2/3, 3) longer protein fragments from carboxyl terminal end of a NPF that include the CA or VCA region, provided the protein is constitutively active. This typically means that the protein is substantially free of at least the GBD. Arp2/3 binding domains also typically do not include the entire amino acid sequence of the NPF.

As noted above, it should also be recognized that Arp2/3 binding domains can be variants of the foregoing sequences (e.g., a CA or VCA sequence or portion thereof) that have substantial sequence identity with the wild type sequence, provided the variant is able to bind an Arp2/3 complex. So, for instance, Arp2/3 binding domains can include proteins that have at least 70, 80, 90, 95, 97, 99% sequence homology with at least 10, 15, 20, 25, 30, 35, 40, 50, 60, 70, 80, 90, 100 amino acids of the C-terminal sequence (e.g., VCA or CA region) of a NPF such as those listed in Table 1. Sequence homology can also extend over the entire region of the sequence (e.g., the entire CA or VCA region).

2. Linkers/Tags

Some affinity ligands are fusion proteins in which the Arp2/3 binding domains is fused to one or more tags at the amino and/or carboxyl terminal ends of the binding domain. The tags can be utilized to improve expression, to improve solubility, to aid in purification and/or to serve as a linker that becomes attached to the affinity matrix. A variety of tags can be utilized. Tags that can be utilized include, but are not limited to, 1) a glutathione S-transferase (GST) tag, which binds to glutathione-agarose; 2) a His6 tag (or simply HIS tag) that binds to immobilized metal-ion columns; 3) a calmodulin-binding peptide tag that binds calmodulin-agarose columns; 4) an epitope tag (e.g., haemagglutinin, myc and FLAG tags) that is bound by an antibody with specific binding affinity for the epitope tag; and 5) a maltose-binding protein, which increases the solubility of fused proteins. These tags can also be used in combination, with one or more tags fused to the amino terminus and one or more additional tags fused to the carboxyl terminus.

Tags such as these can optionally be linked to segments that include protease cleavage sites to remove the purification tag and simultaneously elute the proteins. An example are fusion proteins in which the Arp2/3 binding domain is fused to a tobacco etch virus (TEV) protease site linker and a tag such as protein A. The protein A domain binds tightly to immunoglobulin-gamma columns. Bound Arp2/3 can be released by exposing the column to a highly specific TEV protease.

Fusion proteins containing one or more tags can be prepared using conventional molecular biological techniques such as described in Sambrook et al. Molecular Cloning: A Laboratory Manual, 3rd ed., Cold Spring Harbor Press, N.Y. (2001); and Current Protocols in Molecular Biology (Ausubel, F. M., et al. eds.) John Wiley & Sons, Inc., New York (1987-1993), which are incorporated herein by reference in their entirety for all purposes.

3. Support Material

A variety of support materials can be utilized in the production of the affinity matrix. Examples of suitable materials include, but are not limited to, agarose materials and cellulose materials, including the Sepharose materials available from Amersham Pharmacia Biotech. Many of the affinity matrix materials that are commercially available are functionalized (i.e., contain active functional groups) to facilitate attachment of ligands such as those including the Arp2/3 protein domain to the support material. One example of such a supplier is Amersham Pharmacia Biotech. Guidance regarding the selection and preparation of affinity matrices is provided, for example, in Affinity Chromatography: Principles and Methods, Amersham Pharmacia Biotech AB, 2001.

4. Exemplary Affinity Matrices

One example for an affinity matrix that incorporates some of the foregoing elements is a fusion protein that includes the VCA region from WASP. The amino terminus is fused to a glutathione-S-transferase (GST) tag, and the carboxyl terminus fused to a HIS tag. The generalized sequence of the resulting fusion protein is thus GST-VCA-His. Details regarding the expression of this fusion protein, its purification and its coupling to a Ni-NTA (Qiagen) are provided in Example 2. In general, however, the GST-VCA-His fusion protein is overexpressed in E. coli. The cells are lysed and the fusion protein separated by running the cell extract through a glutathione-Sepharose column (Amersham Pharmacia Biotech), with the GST tag of the fusion protein binding to the glutathione ligands on the column. Purified fusion protein is eluted from the glutathione-Sepharose column and then bound to a Ni-NTA column, with the His6 tag binding to the nickel within the matrix of this column. The resulting Ni-His6-VCA-GST affinity matrix can then be utilized as the matrix to further purify Arp2/3 contained in the fractions collected from the second anion exchange chromatography procedure.

Similar fusion proteins can be prepared for use as a ligand on an affinity matrix. So, for example, the CA region or the VCA region of the NPFs listed in Table 1 can be fused to a GST tag and/or a His6 tag. Examples thus include, but are not limited to, 1) GST-VCA (from N-WASP)-His6; 2) GST-VCA (from WAVE 1)-His6; 3) GST-VCA (from WAVE 2)-His6; and GST-VCA (from WAVE 3)-His6. Other tags such as those listed above, can be utilized in place of the GST and/or His tags. The GST and His6 tags listed in these exemplary constructs could also be reversed so the GST tag is at the carboxyl terminus and the His6 tag at the amino terminus.

IV. Arp2/3 Assays

A number of assays are available to assay for the presence of Arp2/3 during the purification procedure (e.g., identifying fractions containing Arp2/3). One option is a kinetic ELISA protocol (see, e.g., Kelleher, et al. (1998) Methods of Enzymology 298:42-51, incorporated herein by reference in its entirety for all purposes). Another option is to monitor polymerization at the cell surface of the bacterium Listeria monocytogenes (see, e.g., Welch and Mitchison (1998) Methods of Enzymology 298:52-61, incorporated herein by reference in its entirety for all purposes). Other Arp2/3 assay methods are discussed by Egile, et al. (J. Cell Biol. 146:1319-1332, 1999, incorporated herein by reference in its entirety for all purposes).

Another assay is one in which the polymerization of pyrene-labeled actin is monitored. The assay is based upon the observation that as monomeric or globular pyrene-labeled actin (pyrene-G actin) is polymerized to form filamentous pyrene-actin (pyrene-F actin) there is a significant increase in pyrene fluorescence. Because Arp2/3 is required for polymerization to be initiated, the increase in pyrene fluorescence associated with actin polymerization can thus be used as a measure of the presence of Arp2/3.

The assay generally involves combining the necessary elements for actin polymerization, but initially without Arp2/3. The basic elements of the assay include actin, pyrene-actin, a constitutively active form of Cdc42 and a soluble WASP construct. A sample potentially containing Arp2/3 obtained during the purification process is added to the assay mixture. In such an assay mixture, Cdc42, which is an upstream regulator of WASP, activates the WASP construct. The activated WASP construct in turn binds to Arp2/3 in the sample, if present, and activates the complex. Any activated Arp2/3, can then initiate polymerization of pyrene actin into filaments. As polymerization continues and pyrene-actin becomes incorporated into filamentous actin, an increase in fluorescence is detected. Additional details regarding this particular assay approach are set forth in Example 4. This assay is described in even greater detail in U.S. Provisional Application No. 60/578,949, filed Jun. 10, 2004, which is incorporated herein by reference in its entirety for all purposes.

V. Purified Arp2/3 Compositions

Purified Arp2/3 compositions in which the subunits are present in equal stoichiometry are provided. In such compositions, the stoichiometry of the subunits is balanced; thus, for example, the molar concentration of each of the subunits relative to the other subunits is essentially 1:1. Some compositions are also characterized by the p40 subunit, which is particularly susceptible to degradation, being present in an active form without degradation.

The purity of the Arp2/3 complex in the compositions that are provided is typically at least 80, 85, 90, 95, 97, 99 or 100%. These purity levels are on a weight/weight basis and are determined relative to the other proteins that are in solution. So, for example, for a composition having an Arp2/3 purity of 95%, Arp2/3 accounts for 95% of the total protein in the sample on a weight/weight basis.

Some compositions also include various stabilizing agents such as glycerol. Potassium chloride is also present in some solutions. A common storage medium contains about 30% glycerol (v/v) and about 20-40 mM KCl (e.g., 30 mM KCl).

Some of the compositions contain no or only low concentrations of magnesium ion since this can dramatically effect actin polymerization. Thus, in some compositions, the magnesium ion concentration is less than 0.5-1.5 mM, or less than 1 mM. Some compositions are substantially free of magnesium ion, which generally means that the magnesium concentration is below detection level.

VI. Exemplary Applications

As noted in the Background section, actin polymerization is a key aspect in many cellular processes such as cell motility, changes in cell shape and cellular uptake of external agents. Some pathogens also utilize host actin assembly processes to enter cells and/or spread from cell to cell. Arp2/3 as the complex that initiates actin nucleation thus plays an important role in such processes. As also noted above, regulation of Arp2/3 is a complex process that involves various NPFs (e.g., WASP, N-WASP and SCAR/WAVE) and upstream regulatory agents (e.g., Cdc42 and PIP₂) that regulate the activity of the NPFs.

The purification procedures that are provided can thus be utilized to prepare pure Arp2/3 complexes that can be used in a variety of studies on the composition of the complex, its activity and its regulation. Such information can provide additional insight into the foregoing cellular processes. The methods and compositions that are provided, for instance, can be utilized in screening assays to identify compounds that modulate Arp2/3 directly, or the NPFs or upstream agents that directly or indirectly regulate Arp2/3 activity. Some screening methods of this type can be conducted with minor modification of the actin polymerization assays that are provided (see, e.g., Example 3). By including Arp2/3 in the assay composition, assays can be conducted in the presence and absence of a test agent to determine if it is a modulator of actin polymerization. A change in activity in the presence of the rest compound is an indication that the test compound modulates the activity of Arp2/3, a NPF and/or an upstream regulator. Additional details of such screening methods are provided in U.S. Provisional Application No. 60/578,949, filed Jun. 10, 2004, which is incorporated herein by reference in its entirety for all purposes.

The following examples are offered to illustrate certain aspects of the methods and compositions that are described herein. Thus, the examples should not be construed to limit the claimed invention.

### Example 1

### Preparation of Crude Platelet Extract

Platelets are one good source of Arp2/3 because the complex is in relatively high abundance and the total protein concentration low. The proteolytic activity in these cells is relatively low, thus reducing the risk of the complex being degraded during the purification process.

Some preparation methods involve the following process:
1. Outdated platelets were poured into 1 L spinning (centrifuge) bottles.
2. The platelets were spun (centrifuged) at 400 g for about 15 minutes to remove the red blood cells.
3. The supernatant was poured from the bottles into clean 1 L spinning bottles and the supernatant spun at 1200 g for about 15 minutes to obtain pellets of platelets.
4. The supernatant was bleached and then discarded. The remaining pellets that contain the platelets were resuspended in 5-10 ml buffer P [50 mM Tris, pH 7.5, 30 mM NaCl, 10 mM EDTA, 1 mM DTT, protease inhibitors (Roche) (15 tablets per 1 L of crude extract].
5. The resuspended platelets were lysed with a Microfluidizer by running 2 passes, 7-8 cycles each at 80 psi (on the green scale).
6. The lysate was spun in a 45 Ti rotor at 40 Krpm at 4° C. for 2 hours.
7. The supernatant was carefully collected and either dropped frozen in liquid nitrogen or loaded directly on the first anion exchange column (e.g., DEAE) to purify the Arp2/3 in the extract. From 10 L of outdated platelets, 700 ml of lysate with a total protein concentration of about 20 mg/ml was typically obtained.

### Example 2

### Preparation of Affinity Chromatography Material

A. Materials

1. Lysis Buffer:
50 mM Tris; 50 mM KCl; 10 mM Imidazole; 1 mM DTT; pH 7.0.

2. Tris Wash Buffer:
50 mM Tris; 50 mM KCl; 25 mM Imidazole, 1 mM DTT; pH 7.0.

3. Elution Buffer:
50 mM Tris; 300 mM Imidazole; 50 mM KCl; 1 mM DTT; pH 7.4

B. Preparation of Affinity Column Matrix

1. Synthesis and Expression of GST-VCA-His Fusion

WASP full length cDNA is used as a template to amplify the coding sequence. Oligo (forward): 5′-GGGGACAAGTTTGTACAAAAAAGCAGGCTTCGAAAACCTGTATTTTCAGGGCGG GGGTCGGGGAGCGCTTTTGGATC-3′ (SEQ ID NO:6) and oligo (reverse):5′ -GGGGACCACTTTGTACAAGAAAGCTGGGTCCTAGTGATGGTGATGGTGATGGTA GTACGAGTCATCCCATTCATCATCTTCATC-3′ (SEQ ID NO:7) are used in the reaction.

The pcr fragment is cloned into pDONR201 (Invitrogen Life Technology, Cat# 11798-014) by Gateway BP reaction to generate pDONR_tev_WASPVCA_His.

Clone pDONR_tev_WASPVCA_His into pDESTI5 (Invitrogen Life Technology, Cat# 11802-014) to generate N_GST_tev_WASPVCA _His by LR Gateway recombination reaction.

The cloned DNA can be expressed as follows:

Transformation:

Competent cells (BL21 (DE3) or BL21 STAR; Invitrogen) are thawed on ice and approximately 1 μl of DNA is added. Cells are gently mixed and incubated on ice for approximately 30 minutes. After heat shock at 42° C. for 45 seconds, cells are incubated on ice for 2 minutes and 0.5 ml SOC medium is added. Cells are allowed to recover by shaking at 37° C. for one hour, and then plated on selective media (typically LB+100 μg/ml ampicillin).

Day 1

For each new stock test for protein expression:
1. Inoculate several (2-4) 5-10 ml LB-Amp (75 μg/ml Ampicilin) cultures with small fractions of colonies. Mark colonies on a plate to be able to identify mother colony for each culture. Store plate at 4° C. Grow inoculated cultures at 37° C. with shaking until OD₆₀₀=0.8-1. Remove 500 μl sample and collect cells by spinning the sample in an Eppendorf centrifuge 14 Krpm for 2 min; resuspend pellets in 100 μl SDS sample buffer.
2. Add IPTG to 0.5 mM to the remaining culture. Continue growing at 37° C. for 4 hours or at room temperature overnight.
3. Take another set of 500 μl gel samples: collect cells by spinning on an Eppendorf centrifuge 14 Krpm for 2 min; resuspend pellets in 100 μl SDS sample buffer; load 5 μl of each sample on a gel.

Day 2 (or 3)
1. Inoculate 250-500 ml of LB-Amp medium with a single tested colony.
2. Grow at 37° C. with shaking to OD₆₀₀ ˜0.6-0.8.
3. Collect cells by centrifugation on a table top centrifuge at 3 Krpm for 30 mm.
4. Resuspend in 1/10 of initial volume in cold fresh LB-Amp/10%DMSO. Keep cell suspension on ice.
5. Pipette in 1 ml aliquots.
6. Freeze in LN₂. Store at −80° C.

2. Purification of GST-VCA-His Fusion Protein

a. Growth Conditions:
Inoculate culture in the morning with a single fresh colony (use B121(DE3)lysP cells). Use LB medium with (i.e. Sigma T-9179 or Gibco/BRL 22711-022) with 10 ppm antifoam.
Typical volume for a preparation is 1-2 L. Use white baffled flask for 1 L of culture.
Grow at 37° C. with shaking until OD₆₀₀ reaches 1.0-1.2.
Shake at room temperature for 30-45 min.
Add IPTG to 0.5 mM; continue shaking O/N.

b. Harvest cells following morning (after 12-16 hours) by spinning in a bench top Beckman centrifuge at 3 Krpm or in JLA 10 rotor at 5 Krpm for 30 minutes (4° C.). From this point keep solutions on ice and/or at 4° C.

c. Resuspend cell pellets in Lysis buffer supplemented with 1× concentrations of Complete EDTA-free protease inhibitors (Boehringer 1836 170; use 1 mini-tablet per 10 ml) (20 ml for 1 L culture, 40 ml for 2 L). Use dounce homogenizer to make sure resuspension is complete. Proceed with a preparation or freeze cell suspension in liquid N₂ and store at −80° C.

d. Cell Disruption:
When thawing cells, add BME fresh. Lyze cells with the Microfluidizer by running 2 passes, 7-8 cycles each at 80 psi (on the green scale). (If using frozen cells, do 1 pass of 3 cycles). Pass some extra buffer (˜10 ml) through the chamber to rinse it.

e. Spin lysate in 45 Ti at 35 Krpm at 4° C. for 30 min. During this spin pre-equilibrate the resin with lysis buffer (see below).

f. Pre-equilibrate 1.5-2 ml (for 1 L culture) or 3 ml (for 2 L culture) of Ni-NTA resin (Qiagen cat. 31014) with Lysis buffer by washing 2 times with 15 ml of buffer without DTT and protease inhibitors. During these washes collect resin by spinning at 600-700 rpm for 2 min in a bench-top centrifuge.

g. Collect supernatant (save a sample for a gel). Batch load it onto Ni-resin. Incubate at 4° C. for 1 hr with rocking.

h. Pellet the resin by spinning at 600-700 rpm for 2 min. Decant supernatant (save sample for a gel). Resuspend in 5-10 ml of Lysis buffer (with BME and ˜ 1/10 of Complete inhibitors—i.e. 1 mini-tablet per 100 ml) and load resin into a column (use disposable columns or BioRad 1 cm ID EconoColumns). Wash with 50 ml of Lysis buffer. Washes can be done by gravity flow or with a peristaltic pump at 1 ml/min.

i. Pass 10 ml of Tris Wash Buffer through the column.

j. Elute with 81 ml fractions with Elution Buffer with 1/10 of protease inhibitors. Check protein concentrations in fractions by Coomassie Plus (Bradford). Pool peak fractions (protein usually elutes starting at fraction 3).

Measure protein concentration in pooled fractions. Dilute with Tris Wash Buffer+ 1/10 protease inhibitors to 2 mg/ml.

k. Freeze in liquid N₂ by “drop-freezing”. Store at −80° C.

3. Forming Affinity Matrix

The purified GST-VCA-His fusion is coupled to Glutathione-Sepharose (Amersham Biosciences) or related material according to the manufacturer's instructions.

### Example 3

### Purification of Arp2/3 Complex

This example provides a description of one example of the purification of Arp2/3 that incorporates the anion exchange and affinity chromatography procedures (see also FIG. 1).

A. Materials

1. Buffer A:
10 mM TRIS pH 8.0 (room temperature), 1 mM DTT, 1 mM MgCl, 30 mM KCl, 0.2 mM ATP, 1 mM EGTA KOH (0.25M stock pH 7) and 2% Glycerol

2. DEAE Buffer
Buffer A plus 2 tablets of protease inhibitors /1 and 1 mM PMSF.

3. DEAE Chromatography Material (TOYOPEARL DEAE-650M; product #07473; manufactured by Tosh)

4. Q Sepharose Chromatography Material (Q Sepharose Fast Flow; product #17-0510-01, from Amersham Biosciences)

B. Purification Process

1. A cellular extract containing Arp2/3 complex was prepared as described in Example 1.

2. A DEAE column was packed with DEAE material and equilibrated with DEAE buffer. The amount of DEAE material included in the column was calculated based on 250 ml of resin for each 100 ml of crude extract.

3. The conductivity of the extract was adjusted to approximately 30 mM salt (3.6 mS is equivalent to 30 mM salt) and then loaded onto the DEAE column. Flowthrough was collected and the DEAE column washed with about 2 column volumes of DEAE buffer, which was also collected.

4. A Q-Sepharose column was packed and equilibrated with Buffer A. The amount of material was calculated based upon 100 ml of column material for each 200 ml of extract). The collected flowthrough and wash solution was loaded onto the equilibrated column. The column was then washed with 5-10 column volumes of Buffer A containing 30 mM KCl to displace proteins that did not bind or only loosely bound the column material. Bound proteins, including Arp2/3 complex, were subsequently eluted in Buffer A with a salt gradient of 30-300 mM KCl.

5. Fractions containing Arp2/3 were identified by using the actin polymerization method described in Example 4 and active fractions collected. The pooled fractions were diluted to obtain a conductivity of about 3.6 mS.

6. An affinity chromatography column in which the matrix material includes GST-VCA-His6 was prepared as described in Example 2 and equilibrated in Buffer A. Pooled fractions enriched in Arp2/3 complex were then loaded onto the affinity column. The column was washed with about 5 volumes of Buffer A containing 30 mM KCl. Arp2/3 complex was eluted from the affinity column with 250 mM KCl in Buffer A.

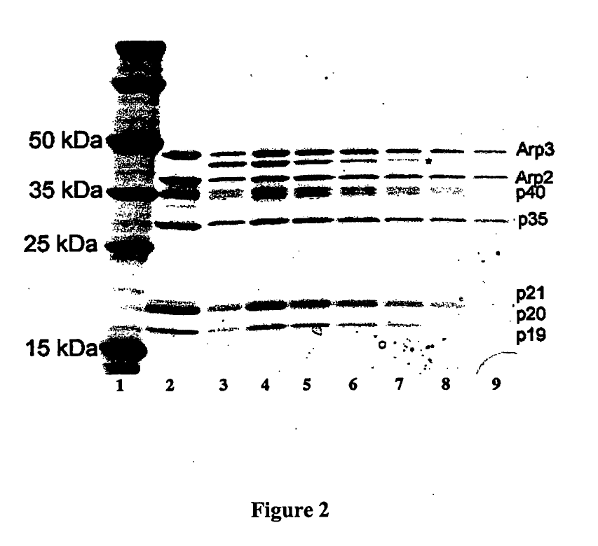

7. Eluted fractions from the affinity column containing purified Arp2/3 were identified using the actin polymerization assay described in Example 4. A gel of fractions eluted from the affinity column is shown in FIG. 2. The lanes in the gel from right to left are: lane 1—protein standards; lane 2—Arp 2/3 complex purified from bovine thymus; lanes 3-9—fractions eluted from the affinity column. The asterisk (*) indicates citrate synthase.

8. Active fractions were concentrated in Y30 Centricons. The purified Arp2/3 was then diluted with fresh Buffer A to obtain a final solution containing about 30 mM KCl. Glycerol was added to about 30% (v/v) and the final protein solution stored at −20° C. The final protein had a purity of at least 95%. Arp2/3 recovery was about 50-60% of the initial Arp2/3 present in the initial extract.

### Example 4

### Actin Polymerization Protocol

A. Materials

G-Actin:

Typically chicken actin was used. G-actin can be purchased from Cytoskeleton, Inc. It can also be purified according to Pardee and Spudich (1982) Methods of Cell Biol. 24:271-89, and subsequently gel filtered as discussed by MacLean-Fletcher and Pollard (1980) Biochem Biophys. Res. Commun. 96:18-27.

Pyrene-Actin:

Typically chicken actin was utilized. Pyrene labeled actin was prepared according to methods described in Kouyama and Mihashi (1981) Eur. J. Biochem. 114:33-38 or as described by Cooper et al. (1983) J. Muscle Res. Cell Motility 4:253-62. Alternatively, it can be purchased from Cytoskeleton, Inc.

GST-Cdc42:
1. pDONR_tev_Cdc42 wt is used as a template for QuickChange site-directed mutagenesis (Stratagene, Cat# 200518). Oligo (forward): 5′-TGTGTTGTTGTGGGCGATGTTGCTGTTGGTAAAACATGT -3′ (SEQ ID NO:8) and oligo(reverse): 5′-ACATGTTTTACCAACAGCAACATCGCCCACAACAACACA-3 ′ (SEQ ID NO:9) are used in this reaction to mutate G12 to a V.
2. Clone pDONR_tev_cdc42GTP into pDESTI 5 (Invitrogen Life Technology, Cat# 11802-014) to generate N-GST-tev-cdc42GTP by LR Gateway recombination reaction.
3. The construct is expressed using the expression protocol listed in Example 2.

GST-105WASP:
1. WASP full length is used as a template to amplify the coding sequence. Oligo (forward): 5′-CACCGAAAACCTGTATTTTCAGGGCCTTGTCTACTCCACCCCCACCCCC-3′ (SEQ ID NO:10) and oligo (reverse):5′-CTAGTCATCCCATTCATCATCT TC-3′ (SEQ ID NO:1 1) are used in the reaction.
2. The pcr fragment is cloned into pENTR/SD/TOPO vector (Invitrogen Life Technology, Cat# K2400-20) by directional cloning using Topoisomerase 1.
3. The pENTR/SD/TOPO_{—}105LWASP is cloned into pDEST15 (Invitrogen Life Technology, Cat# 11802-014) by Gateway LR reaction to generate N_GST_{—}105LWASP.
4. The construct is expressed using the expression protocol listed in Example 2.

Antifoam: Sigma Antifoam

B. Concentration of Stock Reagents and Assay Composition

Arp2/3-mediated Actin Polymerization Protocol

| | | | Assay | |
|---|---|---|---|---|
| Reagents | Concentration | | Conc: | Unit |
| Actin | 0.8 | mg/ml | 3.41 | μM |
| Pyrene-actin | 1.5 | mg/ml | 0.55 | μM |
| GST-Cdc42 | 4.6 | mg/ml | 0.121 | μM |
| GST-105WASP | 0.2 | mg/ml | 0.044 | μM |
| Arp2/3 | 0.3 | mg/ml | 6.6 | nM |
| EGTA | 10 | mM | 55 | μM |
| Antifoam | 2% | | 22 | PPM |
| Number of plates | 35.00 | | | |
| Total Amount Needed | 397.00 | | | |
| First Step: Incubate | | | | |
| CDC-42 with GTP | | | | |
| Thaw appropriate | 588 | μL | | |
| amount ˜ | | | | |
| and add GTP | 65.3224638 | μL | Mix and keep at room | |
| | | | temperature for | |
| | | | 20 min | |
| G-Buffer Total | 265 | mls | Make G-buffer on ice | |
| 10× G-Buffer | 27 | mls | | |
| ATP | 32 | mgs | Add fresh powder | |
| DTT | 133 | μL | | |
| Water | 239 | mls | | |
| Actin Mix (Mix 1) Vol: | 223.5 | mls | Keep this mix on ice | |
| G-buffer | 135.95 | mls | | |
| Actin | 80.02 | mls | 64.01934 | mgs |
| Pyrene-actin | 6.88 | mls | | |
| GST-Cdc42 | 587.90 | μL | | |
| Antifoam | 49.17 | μL | | |
| Arp2/3 Mix (Mix 2) Vol: | 173.50 | mls | | |
| G-Buffer | 130 | mls | | |
| GST-105WASP | 5344 | μL | | |
| Arp2/3 | 1985 | μL | | |
| Antifoam | 38 | μL | | |
| EGTA | 1909 | μL | | |

| | | | |
|---|---|---|---|
| 10× Polymerization Salts | 35 | mls | (add last, 400 mM |
| | | | KCl, 8 mM MgCl2, |
| | | | 1× G-buffer |
| | | | w/o DTT, ATP) |

Samples containing candidate agents (individually or as mixtures) are placed into wells on a multi-well plate. Mix 1 is added to each of the wells and mixed with the candidate agent. A sample of Mix 2 is then introduced into each well and the resulting mixture thoroughly mixed. Typically, Mix 1 and Mix 2 are mixed in 1:1 ratio (e.g., 50 μl each of Mix 1 and Mix 2).

Actin polymerization is measured as a function of time by exciting pyrene at 365 nm and by detecting an increase in fluorescence emission at 407 nm. The change in fluorescence over time is utilized to determine a fluorescence parameter (e.g., maximal velocity, time to half maximal fluorescence intensity or area under the curve of a plot of fluorescence versus time.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

TABLE I

| Nucleation | GenBank | SEQ | | | |
|---|---|---|---|---|---|
| Promoting | Accession | ID | CA Protein | VCA Protein | |
| Factor | No. | NO: | Sequence | Sequence | Reference |
| WASP | P42768 | 1 | 455-501 | 429-502 | Winter, et al. |
| | | | | | (1999) Curr. Biol. |
| | | | | | 9: 501-4; and Yarar |
| | | | | | D., et al. (1999) |
| | | | | | Curr. Biol. 9: 555-58 |
| N-WASP | O00401 | 2 | 433-505 | 393-505 | Rohatgi, et al. |
| | | | | | (1999) Cell 97: 221-31 |
| SCAR/WAVE1 | Q92558 | 3 | 491-559 | 492-559 | Welch, et al. (1998) |
| | | | | | Science 281: 105-108 |
| SCAR/WAVE2 | Q9Y6W5 | 4 | 431-498 | 431-498 | Machesky et al. |
| | | | | | (2003) Molecular |
| | | | | | Biology of the Cell |
| | | | | | 14: 670-684 |
| SCAR/WAVE3 | Q9UPY6 | 5 | 434-502 | 436-502 | Machesky et al. |
| | | | | | (2003) Molecular |
| | | | | | Biology of the Cell |
| | | | | | 14: 670-684 |
| ActA | | | NA | NA | Welch et al. (1998) |
| | | | | | Science 281: 105-8 |

What is claimed is:

1. A method of purifying an Arp 2/3 complex, comprising:
(a) providing a protein mixture containing the Arp2/3 complex;
(b) flowing the protein mixture through a first anion exchanger under conditions such that the Arp 2/3 complex elutes from the first anion exchanger without binding;
(c) contacting the eluate from the first anion exchanger with a second anion exchanger under conditions such that the Arp 2/3 complex binds to the second anion exchanger;
(d) eluting the Arp2/3 complex from the second anion exchanger;
(e) contacting the eluate from the second anion exchanger with an affinity matrix comprising an immobilized affinity ligand that includes an Arp2/3 complex binding domain that can bind the Arp2/3 complex, whereby the Arp2/3 complex binds to the immobilized affinity ligand; and
(f) eluting the Arp2/3 complex from the affinity matrix to yield purified Arp2/3 complex.

2. The method of claim 1, wherein the protein mixture is an extract obtained from human platelets.

3. The method of claim 2, wherein providing comprises preparing the protein mixture from human platelets by:
(i) centrifuging a sample containing a mixture of platelets and red blood cells to precipitate the red blood cells and obtain a supernatant that contains the platelets;
(ii) centrifuging the supernatant from (i) to form a pellet of platelets;
(iii) resuspending the pellet of platelets in a buffer containing protease inhibitors and having a pH between 7.3-7.8;
(iv) lysing the resuspended pellets to form a lysate; and
(v) centrifuging the lysate to form a lysate precipitate and the protein mixture that contains the Arp2/3 complex.

4. The method of claim 1, wherein the first anion exchanger comprises a tertiary amine group and the protein mixture is flowed through the first anion exchanger at a pH greater than 8.

5. The method of claim 4, wherein the first anion exchanger is a DEAE anion exchanger or an equivalent.

6. The method of claim 1, wherein the second anion exchanger comprises a quaternary amine group.

7. The method of claim 6, wherein the second exchanger is a Q Sepharose anion exchanger or equivalent.

8. The method of claim 6, wherein the Arp2/3 complex is eluted from second exchanger with about a 30-300 mM KCl gradient.

9. The method of claim 1, wherein the Arp2/3 complex binding domain consists of 70-100 amino acids from the C-terminus of a nucleation promoting factor selected from the group consisting of Wiskott-Aldrich syndrome protein (WASP), N-WASP, SCAR/WAVE1, SCAR/WAVE2 and SCAR/WAVE3.

10. The method of claim 1, wherein the Arp2/3 complex binding domain comprises a CA domain from a nucleation promoting factor selected from the group consisting of WASP, N-WASP, SCAR/WAVE1, SCAR/WAVE2 and SCAR/WAVE3.

11. The method of claim 1, wherein the Arp2/3 complex binding domain comprises a VCA domain from a nucleation promoting factor selected from the group consisting of WASP, N-WASP, SCAR/WAVE1, SCAR/WAVE2 and SCAR/WAVE3.

12. The method of claim 1, wherein the VCA domain is from WASP.

13. The method of claim 1, wherein the affinity ligand is a fusion protein in which the Arp2/3 binding domain is fused to one or more tags.

14. The method of claim 13, wherein the affinity ligand is a GST-VCA-His fusion protein.

15. The method of claim 1, wherein the Arp2/3 complex is eluted from the affinity matrix with a buffer solution having a potassium chloride concentration between 230-270 mM.

16. The method of claim 1, wherein the eluate from the first anion exchanger is flowed directly to the second anion exchanger.

17. The method of claim 1, wherein the method is completed in less than 24 hours.

18. The method of claim 1, wherein the stoichiometry of the subunits in the purified Arp2/3 complex is retained.

19. The method of claim 1, wherein the p40 subunit of the purified Arp2/3 complex retains its structural integrity.

20. The method of claim 1, wherein elution of the Arp2/3 complex from the first anion exchanger, the second anion exchanger and the affinity matrix is achieved with buffers that contain no more than 1 mM magnesium ion.

21. The method of claim 1, wherein the purified Arp2/3 complex is obtained in at least 50% yield relative to the Arp2/3 complex concentration in the protein mixture.

22. The method of claim 1, wherein the purified Arp2/3 complex is at least 95% pure.

23. The method of claim 1, wherein
(i) the protein mixture is a platelet extract containing the Arp2/3 complex;
(ii) the first anion exchanger is a DEAE anion exchanger or equivalent and the protein mixture is flowed through the DEAE anion exchanger or equivalent at a pH greater than 8;
(iii) the second anion exchanger is a Q Sepharose anion exchanger or equivalent and the Arp2/3 complex is eluted from the Q Sepharose anion exchanger or equivalent with potassium chloride gradient;
(iv) the affinity ligand comprises a VCA domain from a nucleation factor protein;
(v) the Arp2/3 complex is eluted from the affinity matrix with buffer solution containing between 230-270 mM potassium chloride; and
(vi) the stoichiometry of the subunits in the purified Arp2/3 complex is balanced.

24. A purified preparation of Arp2/3 complex characterized by stoichiometric representation of its subunits.

25. The purified preparation of claim 24, wherein the p40 subunit of the purified Arp2/3 complex is undegraded.

26. The purified preparation of claim 20, wherein the Arp2/3 complex is at least 95% pure.

27. An affinity column matrix comprising a support matrix and an affinity ligand comprising a GST domain, a VCA domain and a His tag domain.

28. The affinity column matrix of claim, wherein the VCA domain is from WASP.


SEQUENCE LISTING (11 sequences)

SEQ ID NO: 1 - protein, 502 aa, Homo sapiens
MSGGPMGGRPGGRGAPAVQQNIPSTLLQDHENQRLFEMLGRKCLTLATAVVQLYLALPPGAEHWTKEHCGAVCFVKDNPQKSYFIRLYGLQAGRLLWEQELYSQLVYSTPTPFFHTFAGDDCQAGLNFADEDEAQAFRALVQEKIQKRNQRQSGDRRQLPPPPTPANEERRGGLPPLPLHPGGDQGGPPVGPLSLGLATVDIQNPDITSSRYRGLPAPGPSPADKKRSGKKKISKADIGAPSGFKHVSHVGWDPQNGFDVNNLDPDLRSLFSRAGISEAQLTDAETSKLIYDFIEDQGGLEAVRQEMRRQEPLPPPPPPSRGGNQLPRPPIVGGNKGRSGPLPPVPLGIAPPPPTPRGPPPPGRGGPPPPPPPATGRSGPLPPPPPGAGGPPMPPPPPPPPPPPSSGNGPAPPPLPPALVPAGGLAPGGGRGALLDQIRQGIQLNKTPGAPESSALQPPPQSSEGLVGALMHVMQKRSRAIHSSDEGEDQAGDEDEDDEWDD

SEQ ID NO: 2 - protein, 505 aa, Homo sapiens
MSSVQQQPPPPRRVTNVGSLLLTPQENESLFTFLGKKCVTMSSAVVQLYAADRNCMWSKKCSGVACLVKDNPQRSYFLRIFDIKDGKLLWEQELYNNFVYNSPRGYFHTFAGDTCQVALNFANEEEAKKFRKAVTDLLGRRQRKSEKRRDPPNGPNLPMATVDIKNPEITTNRFYGPQVNNISHTKEKKKGKAKKKRLTKADIGTPSNFQHIGHVGWDPNTGFDLNNLDPELKNLFDMCGISEAQLKDRETSKVIYDFIEKTGGVEAVKNELRRQAPPPPPPSRGGPPPPPPPPHNSGPPPPPARGRGAPPPPPSRAPTAAPPPPPPSRPSVAVPPPPPNRMYPPPPPALPSSAPSGPPPPPPSVLGVGPVAPPPPPPPPPPPGPPPPPGLPSDGDHQVPTTAGNKAALLDQIREGAQLKKVEQNSRPVSCSGRDALLDQIRQGIQLKSVADGQESTPPTPAPTSGIVGALMEVMQKRSKAIHSSDEDEDEDDEEDFEDDDEWED

SEQ ID NO: 3 - protein, 559 aa, Homo sapiens
MPLVKRNIDPRHLCHTALPRGIKNELECVTNISLANIIRQLSSLSKYAEDIFGELFNEAHSFSFRVNSLQERVDRLSVSVTQLDPKEEELSLQDITMRKAFRSSTIQDQQLFDRKTLPIPLQETYDVCEQPPPLNILTPYRDDGKEGLKFYTNPSYFFDLWKEKMLQDTEDKRKEKRKQKQKNLDRPHEPEKVPRAPHDRRREWQKLAQGPELAEDDANLLHKHIEVANGPASHFETRPQTYVDHMDGSYSLSALPFSQMSELLTRAEERVLVRPHEPPPPPPMHGAGDAKPIPTCISSATGLIENRPQSPATGRTPVFVSPTPPPPPPPLPSALSTSSLRASMTSTPPPPVPPPPPPPATALQAPAVPPPPAPLQIAPGVLHPAPPPIAPPLVQPSPPVARAAPVCETVPVHPLPQGEVQGLPPPPPPPPLPPPGIRPSSPVTVTALAHPPSGLHPTPSTAPGPHVPLMPPSPPSQVIPASEPKRHPSTLPVISDARSVLLEAIRKGIQLRKVEEQREQEAKHERIENDVATILSRRIAVEYSDSEDDSEFDEVDWLE

SEQ ID NO: 4 - protein, 498 aa, Homo sapiens
MPLVTRNIEPRHLCRQTLPSVRSELECVTNITLANVIRQLGSLSKYAEDIFGELFTQANTFASRVSSLAERVDRLQVKVTQLDPKEEEVSLQGINTRKAFRSSTIQDQKLFDRNSLPVPVLETYNTCDTPPPLNNLTPYRDDGKEALKFYTDPSYFFDLWKEKMLQDTKDIMKEKRKHRKEKKDNPNRGNVNPRKIKTRKEEWEKMKMGQEFVESKEKLGTSGYPPTLVYQNGSIGCVENVDASSYPPPPQSDSASSPSPSFSEDNLPPPPAEFSYPVDNQRGSGLAGPKRSSVVSPSHPPPAPPLGSPPGPKPGFAPPPAPPPPPPPMIGIPPPPPPVGFGSPGTPPPPSPPSFPPHPDFAAPPPPPPPPAADYPTLPPPPLSQPTGGAPPPPPPPPPPGPPPPPFTGADGQPAIPPPLSDTTKPKSSLPAVSDARSDLLSAIRQGFQLRRVEEQREQEKRDVVGNDVATILSRRIAVEYSDSEDDSSEFDEDDWSD

SEQ ID NO: 5 - protein, 502 aa, Homo sapiens [sequence omitted]

SEQ ID NO: 6 - DNA, 77 bp, Artificial
  WASP forward primer
ggggacaagtttgtacaaaaaagcaggcttcgaaaacctgtattttcagggcgggggtcggggagcgcttttggatc

SEQ ID NO: 7 - DNA, 84 bp, Artificial
  WASP reverse primer
ggggaccactttgtacaagaaagctgggtcctagtgatggtgatggtgatggtagtacgagtcatcccattcatcatcttcatc

SEQ ID NO: 8 - DNA, 39 bp, Artificial
  GST-Cdc42 forward primer
tgtgttgttgtgggcgatgttgctgttggtaaaacatgt

SEQ ID NO: 9 - DNA, 39 bp, Artificial
  GST-Cdc42 reverse primer
acatgttttaccaacagcaacatcgcccacaacaacaca

SEQ ID NO: 10 - DNA, 49 bp, Artificial
  WASP forward primer
caccgaaaacctgtattttcagggccttgtctactccacccccaccccc

SEQ ID NO: 11 - DNA, 24 bp, Artificial
  WASP reverse primer
ctagtcatcccattcatcatcttc